(19) United States
(12) Patent Application Publication
(10) Pub. No.: US 2013/0205879 A1
(43) Pub. Date: Aug. 15, 2013

(54) Water-Distribution System Comprising a Device for Measuring the Value of at Least One Parameter Representing the Water Quality

(71) Applicants: Arnaud Genin, Maisons-Laffitte (FR); Albin Monsorez, Quimper (FR); Cyrille Lemoine, Courbevoie (FR)

(72) Inventors: Arnaud Genin, Maisons-Laffitte (FR); Albin Monsorez, Quimper (FR); Cyrille Lemoine, Courbevoie (FR)

(73) Assignee: VEOLIA WATER SOLUTIONS & TECHNOLOGIES SUPPORT, Saint-Maurice Cedex (FR)

(21) Appl. No.: 13/701,898

(22) PCT Filed: Jun. 6, 2011
(86) PCT No.: PCT/EP11/59252

§ 371 Date: Apr. 26, 2013

(30) Foreign Application Priority Data
Jun. 4, 2010 (FR) 1054420

(51) Int. Cl. G01N 33/18

(52) U.S. Cl. CPC G01N 33/1886
USPC 73/64.56

(57) ABSTRACT

A water distribution system comprises a water distribution conduit. Disposed in the conduit is a device for measuring a value of at least one parameter representative of the quality of the water flowing in the conduit. The device comprises means for measuring the parameter and means for directing substantially all of the water through the device in close proximity to the means for measuring the one parameter.

This application is a U.S. National Stage Application of PCT Application No. PCT/EP2011/059252, with an international filing date of 6 Jun. 2011. Applicant claims priority based on French Patent Application No. 1054420 filed 4 Jun. 2010. The subject matter of these applications is incorporated herein.

### 1. FIELD OF THE INVENTION

The field of the invention is that of controls on the quality of waters flowing in a distribution network.

More specifically, the invention pertains to the designing and making of devices implemented to measure the quality of these waters.

### 2. PRIOR ART

Water-treatment methods are currently implemented for example in order to make it potable, purify it and desalinate it.

The treated waters produced by the application of these methods are led to their distribution point through a network of conduits.

The quality of the treated water is generally controlled directly at the outlet of the treatment units implemented to produce this water. It can then be known if the water produced has a level of quality adequate for its distribution. The distribution of treated water can thus be interrupted if it is detected that it is not of a suitable quality.

However it can happen that the quality of a treated water deteriorates between the outlet of the treatment unit from which it comes and its point of distribution. This may lead to the distribution of treated water of lower quality.

To overcome this drawback, devices for measuring the quality of water have been designed to be set up no longer at the outlet of the water-treatment units but directly within the network of conduits and preferably in proximity to the distribution points.

A device for measuring the quality of a water generally comprises a probe having a body, the head of which is provided with one or more means for measuring capable of measuring parameters representing the quality of a water such as for example its chlorine content, its temperature, its turbidity, etc. The body of this type of probe is introduced into a bypass conduit connected to a main water-distribution conduit for distributing treated water so in such a way that its head carrying a means for measuring bathes in the water flowing therein. The bypassed water is reintroduced into the main conduit or else it is discarded. A technique of this kind is described in the Japanese patent JP-A1-2008058024.

This technique for measuring by bypass then has the advantage of making it possible to know the quality of the produced water when it is at its distribution point or at least close to its distribution point.

However, the technique in which the bypassed water is reintroduced into the main conduit dictates the use, for this purpose, of costly and energy-intensive means or else makes it necessary to give the bypass conduit a particular geometry causing a risk of reducing the speed in the bypass conduit or even creating a “dead arm”, i.e. an area in which the speed of circulation of the water is zero or almost zero, thus falsifying the measurement.

Besides, the water losses caused by the application of the technique in which the bypassed water is discarded into the natural environment, for example a river, a lead to drop in productivity and a risk of retro-pollution. The phenomenon of retro-pollution consists in the injection, through the bypass conduit, of water coming from the natural environment into the main conduit. Water from the natural environment is then mixed with the mains water flowing in the main conduit, thus causing the quality of this network water to deteriorate.

In order to overcome these drawbacks, it has been proposed in the prior art to directly introduce the body of the probe of such a device into a conduit for distributing treated water so that its head, carrying measurement means, bathes in the water flowing therein. A technique of this kind is described for example in the international patent application WO-A1-2007/049003.

This implementation which also makes it possible to know the quality of the water produced when it is at its distribution point or at least close to its distribution point nevertheless suffers from a few drawbacks.

### 3. DRAWBACKS OF THE PRIOR ART

This prior-art technique has one drawback in particular related to the fact that only a reduced portion of the treated water flowing in a distribution conduit into which a probe is introduced passes so that it faces the head carrying the measurement means. The result of this is that the measurements made by means of the probe do not perfectly represent the real quality of the water.

Another drawback of this prior-art technique is related to the fact that certain measurement means implemented in this prior-art technique consume the species whose concentration they measure. Thus, certain chlorine-means for measuring consume the chlorine present in the water when they measure its concentration. It can therefore happen that the local concentration of the water in the species measured is lower in proximity to the measurement means than its real concentration in the water flowing in the conduit. The measurement made is then poorly representative of reality if the renewal of the water to be analyzed is low in the vicinity of these measurement means.

The heads of these prior-art probes also tend to get fouled over time. The quality of the measurements made by their application therefore tends to gradually diminish. It is then necessary to regularly dismantle them in order to clean them.

These prior-art probes generally are relatively large-sized in diameter as well as length which range respectively from 35 to 60 mm and 30 to 1000 mm. However, many distribution conduits have a small nominal diameter often ranging from about 15 to 100 mm. It is thus not possible to introduce a prior-art probe into this type of conduit which nevertheless is widely used.

Probes of this type need to be powered with electrical energy in order to work. They are generally situated in places where it is not possible to connect them to the electrical mains network. These probes are then powered with electrical energy by means of batteries housed in their body. These batteries must be regularly replaced so as to ensure the efficient working of the probes. These probes are nevertheless positioned in places that are difficult to access, and this may make it difficult to replace their batteries. Moreover, it can happen that the batteries powering a probe get discharged without being replaced, leading to a situation where controls are no longer made on the quality of the water. The controls on the distributed water are then no longer done.

### 4. GOALS OF THE INVENTION

The invention is aimed especially at overcoming these drawbacks of the prior art.

More specifically, it is an goal of the invention, in at least one embodiment, to provide a technique for controlling at least one parameter representing the quality of a water, the implementation of which makes it possible to have a piece of information on the quality of the water that is representative of reality.

It is another goal of the invention, in at least one embodiment, to implement a technique of this kind that can be implemented within a network for distributing treated water, the conduits of which are small in size.

It is yet another goal of the invention, in at least one embodiment, to provide a technique of this kind that makes it possible to limit maintenance for the apparatuses used to control the quality of a water.

In particular, the invention is aimed, in at least one embodiment, at providing a technique of this kind that is capable of being implemented for a substantial duration without requiring any intervention.

In particular, in at least one embodiment, the invention is aimed at procuring a technique of this kind that contributes to limiting the fouling of its apparatuses.

The invention is also aimed, in at least one embodiment, at producing a technique of this kind that is not subject to problems related to the supply of electrical energy to its apparatuses.

It is another goal of the invention to offer a technique of this kind that is reliable, robust and simple to implement.

### 5. SUMMARY OF THE INVENTION

These goals as well as others that shall appear here below are achieved according to the invention by means of a device for measuring the value of at least one parameter representing the quality of a water flowing in a water-distribution conduit, said device comprising at least one means for measuring said parameter and means to direct the totality of said water flowing in said distribution conduit so as to be facing said means for measuring.

Thus, the invention relies on a wholly original approach which consists in implementing a device for controlling the quality of a water comprising:
a measurement chamber that houses at least one means for measuring and is to be connected to water inlet and discharge conduits, and
means provided so that all the water flowing from one to the other of these conduits passes so as to be facing these means for measuring.

Thus, as opposed to prior-art probes, the entire volume of the water treated flowing in a conduit of a distribution network travels through a measurement chamber housing one or more means for measuring so that the measurement of the quality of this water is highly representative of its real quality.

Moreover, as opposed to prior-art probes, a device according to the invention is not introduced into a water-distribution conduit. On the contrary, it is inserted between two portions of such a conduit. It can thus be implemented to control the quality of water flowing in small-sized conduits having especially a diameter smaller than that of a probe.

Preferably, said device comprises a measurement chamber housing said means for measuring, said measurement chamber comprising an inlet that is to be connected to a lead-in portion of said water-distribution conduit, and an outlet that is to be connected to a discharge portion of said water-distribution conduit.

According to an advantageous characteristic, a device according to the invention comprises means for generating a turbulent flow of said water facing said means for measuring.

The implementing of this characteristic contributes to limiting the fouling of the means for measuring housed in the measurement chamber by the creation, on their surface, of hydrodynamic stresses tending to prevent the deposition of matter therein and/or to pull away matter that would be deposited therein.

According to a preferred aspect, a device according to the invention comprises means to accelerate the flow of said water facing said measurement means.

The implementing of this characteristic also contributes to limiting the fouling of the means for measuring housed in the measurement chamber by the creation, on their surface, of hydrodynamic stresses tending to prevent the deposition of matter therein and/or to pull away matter that would be deposited therein.

Increasing the speed of flow of the water treated in proximity to the means for measuring causes the local concentration of species present in the water to be very close to the overall concentration of these species in water flowing in the measurement chamber. In this case, when the means for measuring used are of the same type as those that consume the species whose concentration they measure, the speed at which these means for measuring consume these species is lower than their speed of renewal due to the circulation of the water. Implementing this characteristic also enables the improvement, as compared with prior-art probes, of the representativity of the measurement.

The fact of procuring a device, in both these case, for which the fouling speed is considerably reduced limits the frequency at which maintenance campaigns are carried out.

This is particularly useful in as much as the volume of water flowing so that it faces the means for measuring is large as compared with the prior-art techniques.

A device according to the invention advantageously comprises means for converting the hydraulic energy due to the flow of said water in said chamber into electrical energy.

It is then possible to recover energy due to the flow of water in the measurement chamber in order to convert it into electricity which will preferably be used to power the measuring device. This can contribute to increasing the longevity of the batteries that can be used to power the means for measuring or even allow them to function autonomously. This fact thus reduces the maintenance operations at the measuring point.

A device according to the invention preferably comprises means for converting the heat of said water into electrical energy.

It is then possible to recover the heat from the water flowing in the measurement chamber in order to convert it into electricity which will preferably be used to power the device so that it can work autonomously. This characteristic is preferably implemented when the water flowing in the measurement chamber is hot water (preferably at 40 to 80° C.), such as for example domestic hot water.

The fact of being able, in both these cases, to procure device that is autonomous in terms of energy limits the frequency of maintenance campaigns. This again ensures that the device will work permanently.

According to one particular embodiment, a device according to the invention comprises a probe, said probe comprising a body having a head to which said means for measuring are fixedly joined, said body defining, along with the walls of said chamber, a channel for the flow of said water between said inlet and said outlet and passing so as to be facing said head.

The volume of the measurement chamber defined by this channel is then reduced thus improving the representativity of the measurements and eliminating reflow and/or low flow rate regions in this chamber.

According to a preferred characteristic of the invention, said means for generating a turbulent flow comprise a propeller placed between a wall of said chamber and said means for measuring and/or an element for reducing the section of said channel placed between a wall of said chamber and said means for measuring.

The implementation of a propeller of this kind makes it possible, when it is driven by rotation under the effect of the flow of water in the measurement chamber, to create a phenomenon of stirring in proximity to the means for measuring thus limiting their fouling and/or facilitating their cleansing.

The fact that this propeller is driven solely under the effect of the flow of water in the measurement chamber enables the creation of a stirring phenomenon of this kind autonomously without any contribution of external energy.

The application of means for reducing the section of the channel also makes it possible, solely under the effect of the circulation of water in the measurement chamber, to generate a turbulent flow therein.

According to another advantageous characteristic, said means for accelerating the flow comprise an element for reducing the section of said channel placed between a wall of said chamber and said means for measuring and/or a propeller placed between a wall of said chamber and said means for measuring.

Reducing the section of the measurement chamber in proximity to the means for measuring or placing therein a propeller that is free in rotation enables the speed of flow of the water to be increased naturally without any contribution of external energy.

According to a preferred aspect, said means for converting hydraulic energy comprise said propeller, said propeller being mounted so as to be free in rotation within said chamber and connected to at least one magnet, said means for converting hydraulic energy into electrical energy furthermore comprising at least one induction coil placed before said magnet outside said chamber.

Thus, the propeller is connected to magnets that it drives rotationally with respect to a coil placed outside the measurement chamber. Putting the propeller into rotation by the flow of water in the measurement chamber then enables the generation, by induction, of electrical current that could, for example, be accumulated in batteries that are to power the device.

In one variant, said means for converting hydraulic energy comprise said propeller, said propeller being mounted on a shaft mounted so as to be free in rotation within said chamber, one end of this shaft extending outside said chamber and being connected to a current generator.

The first solution described here above in which the propeller is not mounted on a shaft passing through the bottom of the measurement chamber has the advantage of preventing the appearance of leaks between the measurement chamber and the shaft and reducing the dissipation of energy due to the friction of this shaft on the link by which it is connected to the bottom of the measurement chamber.

The device is then autonomous on the energy level and its implementation requires no contribution of external current.

Said means for converting said heat preferably comprise an element made out of thermoelectric material.

This type of material enables efficient conversion of the temperature gradient between the water contained in the conduit and the external environment of this conduit.

In this case, said reduction element is at least partly covered with said thermoelectric material.

The present invention also covers a measurement chamber for a device for measuring the value of at least one parameter representing the quality of a water according to the invention.

Such a measurement chamber comprises an inlet that is to be connected to a lead-in portion of said water-distribution conduit, and an outlet that is to be connected to a discharge portion of said water-distribution conduit and a receptacle that is to house a probe comprising a body and a head to which there are fixedly joined at least one means for measuring said parameter, the walls of said chamber defining, with said body, when said probe is housed in said receptacle, a channel for the flow of said water between said inlet and said outlet and passing so as to be facing said head.

The present invention also covers water-distribution plant comprising a water-distribution conduit and a device for measuring the value of at least one parameter representing the quality of a water flowing in said distribution conduit according to any one of the variants described here above.

### 6. LIST OF FIGURES

Other features and advantages of the invention shall appear more clearly from the following description of preferred embodiments given by way of illustratory and non-exhaustive examples and from the appended drawings, of which:

FIG. 1 schematically represents a view in section of a device according to the invention implementing a ramp;

FIG. 2 illustrates a view in perspective of the device illustrated in FIG. 1;

FIG. 3 schematically represents a view in section of a device according to the invention implementing a propeller;

FIG. 4 illustrates a view in perspective of the device illustrated in FIG. 3;

FIG. 5 illustrates a variant of the device of FIGS. 3 and 4;

FIG. 6 illustrates a view of a measuring device according to the invention mounted on a water-distribution conduit at the outlet of a treatment plant.

### 7. DESCRIPTION OF ONE EMBODIMENT OF THE INVENTION

### 7.1. Reminder of the Principle of the Invention

The general principle of the invention consists in implementing a device for controlling the quality of a water that comprises:
a measurement chamber that houses at least one means for measuring and is to be connected to water inlet and discharge conduits, and
means provided so that all the water flowing from one to the other of these conduits passes so as to be facing these means for measuring.

As opposed to prior-art probes, the entire volume of treated water flowing in a conduit of a distribution network thus travels through a measurement chamber housing one or more means for measuring. The result of this is that the measurement of the quality of this water is highly representative of its real quality.

Besides, a device according to the invention can thus be implemented to control the quality of water flowing in small-sized conduits because it is intended for insertion between two portions of a water-distribution conduit.

### 7.2. Example of a First Embodiment of the Invention

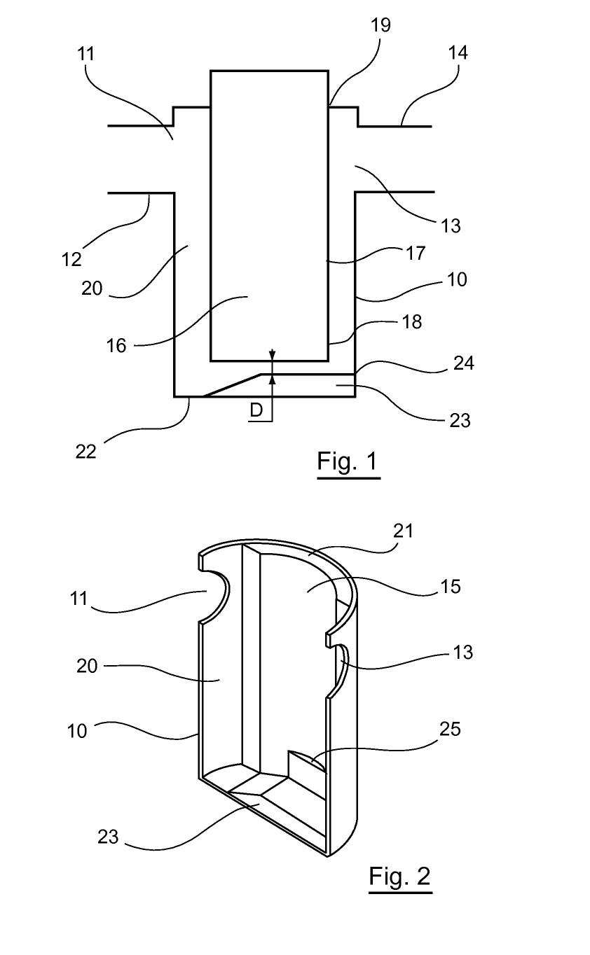

Referring to FIGS. 1 and 2, we present a first embodiment of a device for measuring the value of at least one parameter representing the quality of a water according to the invention.

As represented in FIGS. 1 and 2, such a device comprises a measurement chamber 10. In this embodiment, this measurement chamber 10 has a circular section and takes the form of a hollow cylinder.

The measurement chamber 10 has an inlet 11 to be connected to a water lead-in conduit 12 and an outlet 13 to be connected to a pipe 14 for discharging this water.

The device according to the invention is planned for installation between two portions of a water-distribution conduit. The lead-in conduit is therefore a lead-in portion of the distribution conduit and the discharge conduit is a discharge portion of the distribution conduit.

The measurement chamber 10 defines a receptacle 15 capable of housing a probe 16 comprising a body 17 and a head 18 to which there are fixedly joined means for measuring (not shown) provided in order to measure parameters representing the quality of the water flowing in the measurement chamber 10.

The measurement chamber 10 comprises an aperture 19 to enable the probe 16 to be housed in the receptacle 15.

When a probe 16 is housed in the receptacle 15, its body 17, along with the inner walls of the measurement chamber 10, defines a channel 20 for the flow of water passing through between the inlet 11, the head 18 of the probe 15 and the outlet 13.

Lateral stops 21 are interposed on either side of the body 17 of the probe 16 between the walls of the measurement chamber 10 and the body 17. Their dimensions are chosen so that the water flowing in the measurement chamber 10 cannot flow around the probe 16 but on the contrary is forced to pass beneath the head 18 of the probe 16.

The bottom of the measurement chamber 10 houses means to accelerate the flow of water and generate a turbulent flow facing the means for measuring fixedly joined to the head 18. These means comprise an element for reducing the section of the channel 20 placed between the bottom 22 of the measurement chamber 10 and the means for measuring. This element for reducing comprises a ramp-forming element 23.

This device according to the invention furthermore comprises means for converting the temperature gradient between the water flowing in the measurement chamber 10 and the external environment into electrical energy. These means for converting comprise a thermoelectric material 24 which partly covers the ramp. In one variant, this thermoelectric material 24 will totally cover the ramp. This material is connected to batteries (not shown) used to supply the probe 16 with electrical current.

Each stop 21 has a lower part forming an end stop 25 against which the head 18 of the probe 16 rests so that it is situated at a distance “D” of 1 mm to 10 cm from the surface of the ramp.

### 7.3. Example of a Second Embodiment of the Invention

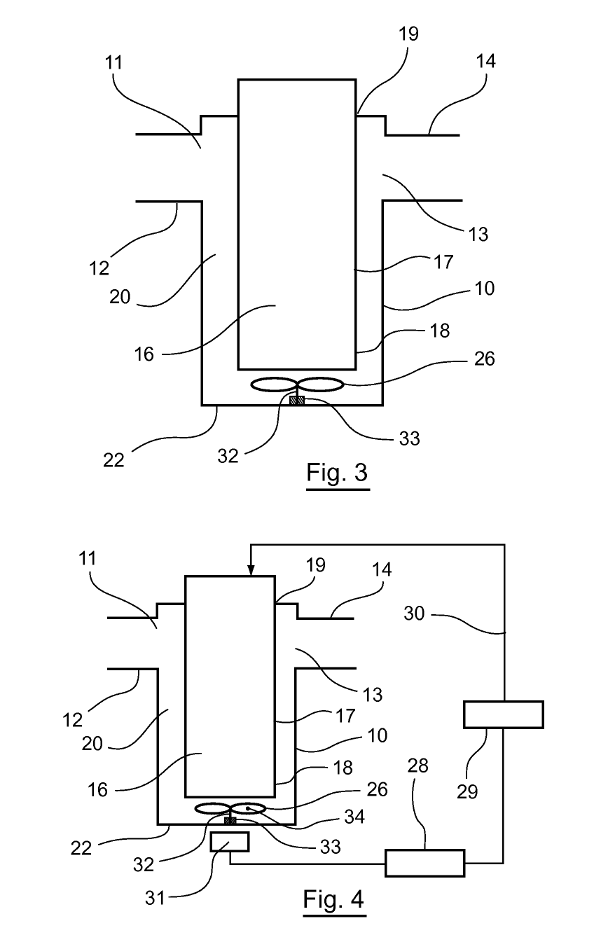

Referring to FIGS. 3 and 4, we present a second embodiment of a device for measuring the value of at least one parameter representing the quality of a water according to the invention.

This second embodiment has a large number of similarities with the first embodiment described further above.

More specifically, this second embodiment can be distinguished from the first one by the fact that it does not implement means for converting the heat of the water flowing in the measurement chamber 10 into electrical energy.

One device according to this second embodiment comprises, on the contrary, means for converting the hydraulic energy due to the flow of water in the measurement chamber 10 into electrical energy.

Furthermore, the means for accelerating the generation of a turbulent flow comprise no longer an element for reducing the section but a propeller 26 which is placed between the bottom 22 of the measurement chamber 10 and the means for measuring. This propeller 26 is fixedly joined to a shaft 32 that is essentially perpendicular to the bottom 22 of the measurement chamber 10 and mounted so as to be free in rotation in a bearing 33 fixedly joined to this bottom 22. In one variant, the propeller 26 could be fixedly joined to a shaft mounted so as to be free in rotation in a bearing fixedly joined to the head 18 of the probe.

The means for converting the hydraulic energy from the flow of water in the measurement chamber 10 into electrical energy comprise this propeller 26. They furthermore comprise magnets 34 fixedly joined to the propeller 26 and a coil 31 placed outside the measurement chamber facing the magnets 34. In one variant, the magnets could be carried by a part fixedly joined to the shaft 32.

The magnets 34 and the coil 31 supply electrical current through a charge regulator 28 to the batteries 29 when the propeller 26 is driven rotationally under the effect of the flow of water in the measurement chamber 10. The batteries 29 are connected to the probe 16 by electrical cables 30 to ensure its operation.

The distance “d” between the surface of the means for measuring and the upper part of the propeller 26 ranges from 1 to 20 mm.

As shown in FIG. 6, a measuring device 62 according to the invention, whatever the form in which it is made, is intended for mounting on a conduit for distributing water between the outlet of a water treatment plant 60 and a water-distribution point 61. The water-distribution conduit comprises a lead-in conduit or portion 12 and a discharge conduit portion 14 respectively connected between the inlet 11 and the outlet 13 of the measurement chamber of the measuring device 62. All the water flowing in the distribution conduit therefore travels through the measurement chamber of the measuring device without any bypass.

### 7.4. Variants

In variants, a device according to the invention could comprise:
means to accelerate the flow of water facing the means for measuring; and/or
means for generating a turbulent flow of water facing the means for measuring;
and/or
means for converting the temperature gradient between the water flowing in the measurement chamber 10 and the external environment into electrical energy; and/or
means for converting the hydraulic energy due to the flow of water in the measurement chamber 10 into electrical energy.

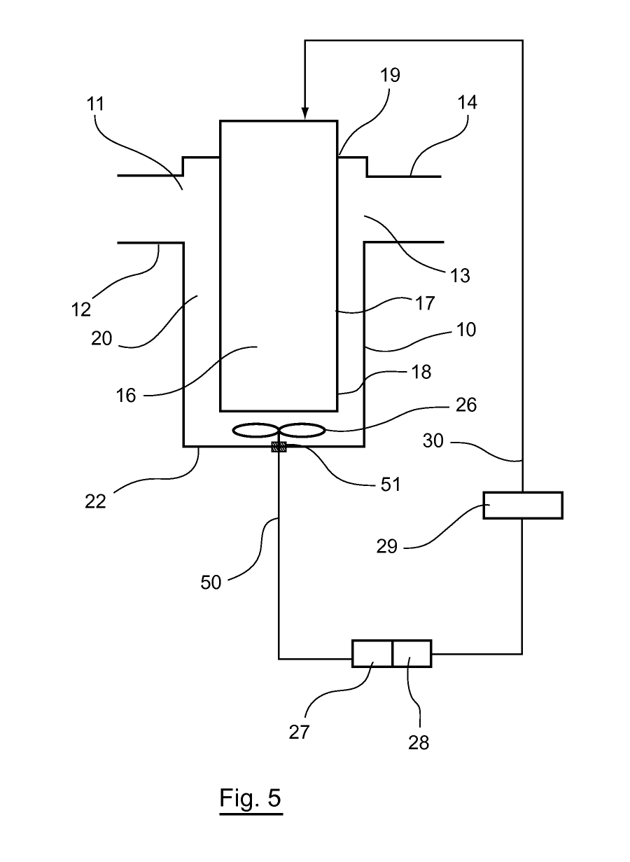

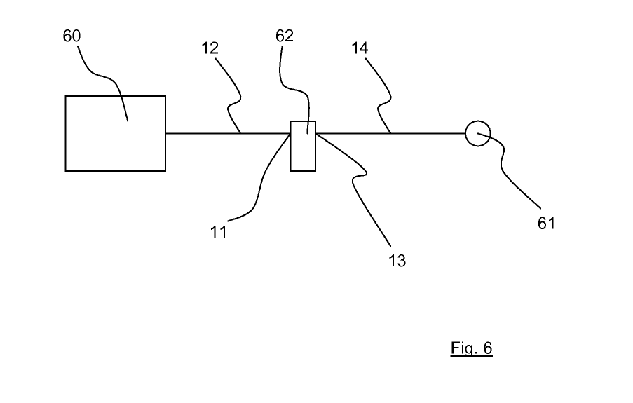

In one variant illustrated in FIG. 5, the propeller 26 is fixedly joined to a shaft 50 having one end crossing the bottom 22 of the measurement chamber through which it is mounted so as to be free in rotation by means of a tightly sealed bearing 51. This end of the shaft 50 is mechanically connected to a generator 27. The generator 27 provides electrical power through a charge regulator 28 to the batteries 29 when the propeller 26 is driven in rotation under the effect of the flow of water in the measurement chamber 10. The batteries 29 are connected to the probe 16 by electrical cables 30 to ensure its operation.

### 7.5. Trials

Trials consisted in making the water flow into a measurement chamber of a device according to the invention:
housing neither a ramp nor a propeller;
housing a ramp;
housing a propeller.

In these trials, the water flowed at a flow rate equal to 500 l/h in a measurement chamber with a volume equal to 25 cm³. The distance “D” between the head 18 of the probe 16 and the surface of the ramp was equal to one centimeter. The distance between the surface of the means for measuring and the upper part of the propeller was also equal to one centimeter.

The speed of flow of water at one millimeter from the means for measuring was equal to:
0.6 m.s-1 without a ramp or propeller;
1 m.s-1 with ramp;
0.7 m.s-1 with propeller.

The speed of flow of the water facing the means for measuring is therefore increased by:
67% through the implementing of a ramp;
17% through the implementing of a propeller.

The turbulent intensity at 1 millimeter from the means for measuring was equal to:
11% without ramp or propeller;
14% with ramp;
12% with propeller.

The turbulent intensity of the water facing the means for measuring is therefore increased by:
27% through the implementing of a ramp;
9% through the implementing of a propeller.

1-13. (canceled)

14. A water distribution system comprising: a water distribution conduit having a lead-in conduit portion and a discharge conduit portion; a water quality measuring device disposed in the water distribution system between the lead-in conduit portion and the conduit discharge portion of the water distribution conduit and including a probe for measuring the value of at least one parameter representing the quality of water flowing in the water distribution conduit; and the water quality measuring device being configured to cause substantially all of the water flowing through the water distribution conduit to flow in close proximity to the water quality measuring device.

15. The water distribution system of claim 14 wherein the water quality measuring device comprises a measurement chamber housing the probe for measuring the quality of at least one parameter representing the quality of water flowing in the water distribution conduit and wherein the measurement chamber comprises an inlet connected to the lead-in conduit portion of the water distribution conduit and an outlet connected to the discharge conduit portion of the water distribution conduit.

16. The water distribution system of claim 1 further including means for generating a turbulent flow of water adjacent the probe.

17. The water distribution system of claim 16 wherein the means for generating the turbulent flow comprises a water flow restrictor disposed in the path of the water flowing through the water quality measuring device.

18. The water distribution system of claim 17 wherein the flow restrictor comprises a ramp disposed in the path of water flow through the water quality measuring device.

19. The water distribution system of claim 16 wherein the means for generating a turbulent flow comprises a propeller disposed in the path of the water flow through the water quality measuring device.

20. The water distribution system of claim 14 including means for accelerating the flow of water passing adjacent the probe.

21. The water distribution system of claim 20 wherein the means for accelerating the flow of water passing adjacent the probe comprises a ramp disposed in close proximity to the probe and wherein substantially all of the water flowing through the water distribution conduit is constrained to move between the ramp and the probe.

22. The water distribution system of claim 14 including means for converting the hydraulic energy due to the flow of the water through the water quality measuring device into electrical energy.

23. The water distribution system of claim 14 including means for converting heat associated with the water passing through the water measuring quality device into electrical energy.

24. The water distribution system of claim 14 wherein the water quality measuring device includes a wall structure and wherein at least a portion of the probe is disposed interiorly of the wall structure and wherein the wall structure and the probe define a channel that constrains water flowing through the water quality measuring device to flow around the probe and through the channel.

25. The water distribution system of claim 24 including a propeller for generating a turbulent water flow adjacent the probe and wherein the propeller is disposed between the wall structure and the probe.

26. The water distribution system of claim 14 including an open channel formed between the probe and a wall that forms a part of the water quality measuring device; and wherein there is provided a water flow restricting device in the channel adjacent the probe.

27. The water distribution system of claim 26 wherein the water flow restriction device comprises a ramp mounted in the channel and which effectively reduces the cross-sectional area of the channel adjacent the probe.

28. The water distribution system of claim 22 wherein the means for converting hydraulic energy into electrical energy comprises a propeller mounted for rotation in a chamber that forms a part of the water quality measuring device and wherein the propeller is operatively associated with at least one magnet and at least one induction coil for generating electrical energy.

29. The water distribution system of claim 28 wherein the induction coil is disposed exteriorly of the water quality measuring device.

30. The water distribution system of claim 14 including a propeller mounted interiorly of the water quality measuring device and particularly disposed to lie at the path of water flowing through the water quality measuring device and wherein the propeller includes a shaft that is operatively connected to a current generator disposed exteriorly of the water quality measuring device.

31. The water distribution system of claim 23 wherein the means for converting the heat of the water into electrical energy comprises a structure constructed of thermoelectric material.

32. The water distribution system of claim 14 wherein there is formed a water flow channel between the probe and a wall structure of the water quality measuring device; and wherein there is provided a flow restriction device in the water flow channel adjacent the probe and wherein the flow restriction device is at least partially covered with a thermoelectric material.

33. A water distribution plant comprising: a water distribution conduit including a device for measuring the value of at least one parameter representing the quality of water flowing in the water distribution conduit, the device including at least one probe for measuring the parameter and wherein the device is configured to direct substantially all of the water flowing in the water distribution conduit through the device such that the water faces the probe.

34. The water distribution system of claim 33 wherein the device includes a chamber formed by a wall structure and wherein the probe is disposed internally within the chamber such that a flow channel is defined between the probe and the wall structure so as to constrain the water flowing through the device to flow through the channel such that a substantial portion of the water flowing through the water distribution conduit is constrained to flow adjacent the probe.

35. The water distribution system of claim 34 where the flow channel includes a series of turns that requires the water flowing through the device to turn and move around the probe as the water moves from an inlet of the device towards an outlet of the device.

36. A method of measuring the quality of water treated in a wastewater treatment facility comprising:
directing treated water from the wastewater treatment facility through a water distribution conduit to an inlet of a water quality measuring device having a chamber and a water quality measuring probe disposed in the chamber;
directing the treated water through the water quality measuring device and through a water flow channel defined between a wall structure of the chamber and the probe such that substantially all of the water passing through the water quality measuring device is constrained to move closely adjacent the probe;
utilizing the probe to determine a value of at least one parameter representing the quality of the water flowing in the water distribution conduit; and
after measuring the parameter representing the quality of the water flowing through the water quality distribution conduit, directing the treated water to an outlet of the water quality measuring device.

37. The method of claim 36 wherein the water flow channel is generally U-shaped and wherein the water flowing through the water quality measuring device flows vertically adjacent the probe and around and across a head of the probe, and then vertically adjacent the probe and out the outlet of the water quality measuring device.

38. The method of claim 36 including generating water turbulence adjacent the probe as the treated water flows through the water quality measuring device.

39. The method of claim 38 including restricting the flow of water passing through the water flow channel so as to give rise to water turbulence adjacent the probe.

40. The method of claim 36 including accelerating the flow of water facing the probe as the water passes through the water quality measuring device.

41. The method of claim 36 including utilizing the hydraulic energy associated with the flow of water through the water quality measuring device to produce electrical energy.

42. The method of claim 35 including converting heat associated with the water flowing through the water quality measuring device to produce electrical energy.
